(19) United States
(12) Patent Application Publication
(10) Pub. No.: US 2015/0025647 A1
(43) Pub. Date: Jan. 22, 2015

(54) DUAL MOBILITY HIP REPLACEMENT SYSTEM

(71) Applicant: Howmedica Osteonics Corp., Mahwah, NJ (US)

(72) Inventor: Zongtao Zhang, Riverdale, NJ (US)

(21) Appl. No.: 13/943,282

(22) Filed: Jul. 16, 2013

(51) Int. Cl. A61F 2/34; A61F 2/36

(52) U.S. Cl. CPC A61F 2/34; A61F 2/3601
USPC 623/22.18

(57) ABSTRACT

A total hip implant system comprising: a prosthetic femoral component having a head with a part-spherical bearing surface; and a dual mobility acetabular cup system comprising a first bearing component made of ultra high molecular weight polyethylene mounted on the head, the first bearing component having a part-spherical inner bearing surface rotatably engaging the part-spherical bearing surface of the head, and having a part-spherical outer bearing surface, a second bearing component made of polyetheretherketone (PEEK) having an inner part-spherical bearing surface rotatably engaging the part-spherical outer bearing surface of the first bearing surface.

### BACKGROUND OF THE INVENTION

Polyaryl ether ketones (PAEK), such as PEEK, which is the most commercialized, are well known polymers such as described in Chapter 37 of the “Handbook of Thermoplastics” published by Marcel Dekker Inc. These polymers are highly aromatic mostly semi crystalline thermoplastics which, because of their aromatic polymer backbone, have transition temperatures as high as 240° C. These polymers may be synthesized by well-known condensation polymerization methods. PAEK has excellent resistance to acids, water and is capable of being sterilized by gamma radiation, ethylene oxide gas and steam.

Reinforced polyetheretherketone (PEEK) has been proposed for us in orthopedic implants such as hip stems and acetabular cups. U.S. Pat. Nos. 5,181,930 and 5,443,513 relate to hip stems made of PEEK including carbon fiber reinforcements. PEEK has also been proposed for use in acetabular cups as either backing or bearing materials. See for example, U.S. Pat. Nos. 6,638,311 and 6,758,864. Flexible acetabular cups made of PEEK have also been proposed as discussed in U.S. Publications 2007/073410 and 2007/0191962. In these publications the opposite bearing, such as a femoral head, have been made of either a ceramic or metal bearing surface.

U.S. Patent Publication 2009/0164023 relates to an all polymeric bearing couple wherein each part is made of a composite material including carbon fiber reinforcement.

PEEK and carbon fiber reinforced PEEK composite as a potential bearing surface for total joint replacement applications was considered in the 1990s (Wang, A., Lin, R., Stark, C., and Dumbleton J H., Wear 225-229 (1999) 724-727). The intention was to replace the ultrahigh molecular weight polyethylene bearing (UHMWPE) with PEEK or carbon fiber reinforced PEEK composite bearings in traditional metal or ceramic-on-UHMWPE bearing couples for total hip and total knee joint replacements. It was found that pure PEEK without carbon fiber reinforcement against a ceramic counterface produced higher wear rate than a traditional ceramic-on-UHMWPE bearing couple while a ceramic-on-carbon fiber reinforced PEEK composite yielded a lower wear rate than a traditional ceramic-on-UHMWPE bearing couple. Metallic bearing counterfaces such as CoCr or stainless steel was found unsuitable against carbon fiber reinforced PEEK composite bearing due to significant scratching of the metallic surface by the harder carbon fibers. Carbon fiber reinforced PEEK-on-PEEK has only been considered for either smaller non-weight bearing or low weight-bearing joints (Qi-Bin Bao, et al, Nubac Disc Arthroplasty: Preclinical studies and preliminary safety and efficacy evaluations, SAS Journal, Winter 2007, Volume 01, issue 01, p. 36-45). A low-to-high wear transition was found for PEEK-on-PEEK as the applied load increased in a wear test study (Heather Austin, et al, Exploring the wear of a peek all-polymer articulation for spinal application, Society for Biomaterials 2009 annual meeting, Apr. 22-25, 2009, San Antonio, Tex.).

PTFE-on-PTFE was first used for total hip replacement by Dr. John Charnley prior to 1962 (Steven M. Kurtz, The UHMWPE Handbook, p. 53-70). Because of poor wear performance; PTFE-on-PTFE has been abandoned. US patent publications 2007/0270970 and 971 relate to polymeric spine bearing components.

Polyacetal-on-Polyethylene was introduced as an all polymer bearing couple in total knee arthroplasty in the 1980's and clinical results were published in the 1990's (1) H. McKellop, et al, Super wear properties of an all-polymer hip prosthesis, 31^{st} Annual ORS, Las Vegas, Nev., Jun., 21-24, 1985, page 322; (2) D. J. Moore, et al. The total knee replacement prosthesis may be made entirely of polymer. The Journal of Arthroplasty, Vol. 13, No. 4, 1998). Because poor gamma sterilization resistance of the polyacetal material (Delrin) and inadequate fixation of the Delrin femoral component to the bone, the use of Polyacetal-on-Polyethylene has been discontinued.

U.S. Patent Application Publication No. 2010/0312348 relates to an orthopedic prosthetic joint couple having a first bearing surface made of non-fiber reinforced PAEK or PEEK and a second bearing surface made of UHMWPE. The disclosure of 2010/0312348 is incorporated herein by reference. PAEK-on-polymer (such as ultra high molecular weight polyethylene (UHMWPE) bearing couples, particularly PEEK (polyetheretherketone) on ultra high molecular weight polyethylene (UHMWPE) have unexpectedly been shown to have excellent wear properties. PAEKs (polyaryletherketones), include PEK (polyetherketone, PEKK (polyetherketoneketone), and PEKEKK (polyarylether-ketoneether-ketone-ketone) and PEEK. If a PEEK bearing is used it can be a stand-alone pure (“neat”) PEEK component (composed entirely of non-reinforced PEEK with no fillers and no other polymers or impurities in a substantial amount), a PEEK layer coated, molded or grafted onto another solid or porous polymer or polymeric composite substrate, or a PEEK layer coated, molded, or grafted onto a solid or porous metallic or ceramic substrate. The polymer bearing can be any kind of polymer that is softer than the PAEK. The polymer includes but not limited to polyethylene, polyurethane, polyamide, and a composite of these polymers, etc. The polymer may be mono-polymer, co-polymer, surface grafted polymer or coated polymer. More specifically, this invention relates to non-carbon fiber reinforced PEEK-ON-UHMWPE as a bearing couple for orthopedic applications. The bearings are used in artificial joints that replace biological joints such as hips, knees, shoulders, elbows, fingers and spine.

This invention uses neat (un-reinforced especially non-carbon fiber reinforced) PEEK, or a PAEK polymer with similar properties, to replace the typical metal or ceramic as one of the bearing surfaces in a metal-on-polymer or ceramic-on-polymer pair. It has unexpectedly been found that PEEK-on-polymer bearing couples (such as PEEK-on-polyethylene) have lower wear rates than typical orthopedic bearing couples (such as metal-on-UHMWPE). The mechanism of the low wear rate of PEEK-on-polymer may be contributed to two mechanisms. (1) Less total contact stress. Since the PEEK has a much lower Young's modulus than cobalt chrome molybdenum alloy (CoCr), the PEEK has high elastic deformation under the same compressive force, which may facilitate elastohydrodynamic lubrication than conventional metal or ceramic on polymer joints. (2) Local sharpness effect: The wear takes place when two surfaces contact and rub each other. The wear rate is highly determined by the sharpness and hardness of the surface asperities under standard body contact force and wet lubrication. PEEK has very low hardness (about Shore D 85) as compared to CoCr alloy (Vickers 450), thus the asperities of PEEK are blunt and compressible, while the CoCr- is sharp and stiff. The blunt asperities wear the counter surface less than the sharp ones.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

### BRIEF SUMMARY OF THE INVENTION

A polyetheretherketone (PEEK) on ultra high molecular weight polyethylene (UHMWPE) bearing couple has been shown to have lower wear than a similar metal or ceramic on UHMWPE bearing couple in an orthopedic joint simulator. However, certain types of bearing geometries and articulations are better suited to a PEEK-on-UHMWPE bearing couple. One challenge associated with a PEEK-on-UHMWPE bearing is that the PEEK has a lower thermal conductivity than the metal it is replacing. As a result localized heating at the bearing interface can result. (See Baykal D; Rau, A C; Underwood, R J; Siskey, R S; Kurtz S M “Frictional Heating of PEEK-UHMWPE Bearing Couple on Pin-on-Disk Tester” Drexel University, April 2013)

Bearing geometries with relatively more clearance (such as knees) are less prone to increased localized heating since the natural lubricating fluid has greater access to the area of contact between the bearing surfaces as compared to bearings with less clearance (such as hips). Bearing where the area of contact shifts during range of motion (such as knees) are also less prone to increased localized heating as compared to bearings where the area of contact remains relatively constant (such as hips). For these reasons hip simulator testing has shown that using a PEEK head on an UHMWPE acetabular bearing insert produced localized heating.

“Dual mobility” hip replacements such as MDM (Modular Dual Mobility) and ADM (Anatomic Dual Mobility) by Stryker® Corp. are known, and it is known that the “secondary” bearing surface between the fixed shell and mobile bearing insert experiences relatively less articulation than the “primary” bearing surface between the mobile bearing insert and femoral head. Using PEEK for the fixed shell bearing surface offers advantages in terms of cost and wear performance.

Thus the present invention relates to a total hip implant system comprising a femoral component having a part-spherical non-polymeric head (ceramic or metal) having an outer bearing surface and a dual mobility acetabular cup. The dual mobility cup comprises an ultra-high molecular weight polyethylene first bearing component having a part-spherical inner bearing surface and a part-spherical outer bearing surface. The inner bearing surface articulates against the outer bearing surface of the part-spherical head. A second bearing component is composed of substantially non-fiber reinforced pure poly aryl ether ketone polymer selected from the group consisting of polyetherketone (PEIK), polyetherether ketone (PEEK), polyether ketone ketone (PEKK) and polyetherketone etherketoneketone (PEKEKK). The preferred bearing is PEEK. The second bearing component (PEEK) having a part-spherical inner bearing surface articulates against the part-spherical outer bearing surface of the first bearing component (UHMWPE). The second bearing component may have a bone contacting outer surface which may be titanium mesh bone ingrowth surface or metal particles embedded in the PEEK.

The dual mobility cup may also comprise an outer metal shell with an outer bone contacting surface. This outer metal surface may be designed for bone ingrowth. If a metal outer shell is used the second bearing component may be fixedly mounted within the metal shell. For assembling the second bearing component, the outer metal shell may have a circumferentially extending locking element formed on an inner surface for engaging a locking element formed on an outer surface of the second bearing component. The second bearing element may have protrusions or recesses for preventing relative movement between the outer shell and the second bearing component. Preferably the part-spherical non-polymeric head of the femoral component is made from a material selected from the group consisting of titanium, titanium alloy, ceramic, cobalt chrome molybdenum alloy, and stainless steel.

The second bearing component may also have a circumferential distal rim having a generally inferiorly facing edge surface having a contour curved in a generally inferior-superior direction around the entire circumference of the distal rim with the generally inferiorly facing edge surfacing moving towards and away from a plane containing the rim of an acetabulum two times. This inferiorly facing edges surface mimics the natural acetabulum inferior surface.

The goals of a dual mobility hip systems are to increase stability by decreasing the incidence of dislocation while providing excellent wear properties with the use of a larger bearing. Dual mobility polyethylene liners have been shown to have very small angulation of the outer diameter during standard wear testing conditions. Approximately 10% of wear in the dual mobility construct occurs on the outer diameter when CoCr is utilized as the liner material. PEEK is better suited as the larger secondary bearing surface as opposed to the inner/primary bearing surface (which articulates against the femoral component head) because high friction and localized heating may occur when utilizing PEEK as the inner primary articulation. Utilizing PEEK at the outer articulation surface lowers dynamic friction between the PEEK and polyethylene materials compared with the inner articulation. Using PEEK as the secondary bearing keeps the friction and the localized temperature at the bearing surface low and prevents overheating the polyethylene.

### BRIEF DESCRIPTION OF THE DRAWINGS

FIG. 1 shows a prosthetic total hip replacement system including a femoral component and a dual-mobility (bi-polar) acetabular cup system;

FIG. 2 is a cross-sectional view of the acetabular cup system and the proximal end of the femoral component including the prosthetic head as shown in FIG. 1;

FIG. 3 is an exploded view of an alternate embodiment of the present invention which shows a dual mobility acetabular cup system further comprising an outer metal shell for contacting the prepared acetabulum;

FIG. 4 is a partial cross-sectional view of the outer shell shown in FIG. 3;

FIG. 5 shows the assembly of an ultra-high molecular weight polyethylene bearing mounted within a PEEK component having an inner bearing surface which in turn is mounted in the metal shell of FIGS. 3 and 4;

FIG. 6 shows one possible design for the PEEK bearing component having a rim mimicking the rim of a natural acetabulum;

FIG. 7 is an elevation view of the PEEK bearing component shown in FIG. 6; and

FIG. 8 is a table showing the average polyethylene volume loss plotted as a function of cycle count generated between the ultra-high molecular weight polyethylene outer bearing surface and the inner bearing surface of the PEEK component located within a dual mobility acetabular cup system as shown in FIGS. 1 and 2.

### DETAILED DESCRIPTION

Referring to FIG. 1 there is shown a prosthetic total hip system generally denoted as 10 and an acetabular cup system generally denoted as 12 and a femoral component designated as 14. A prosthetic femoral head 13, which is made of metal or ceramic is mounted on a typical trunion at a proximal end of femoral component 14.

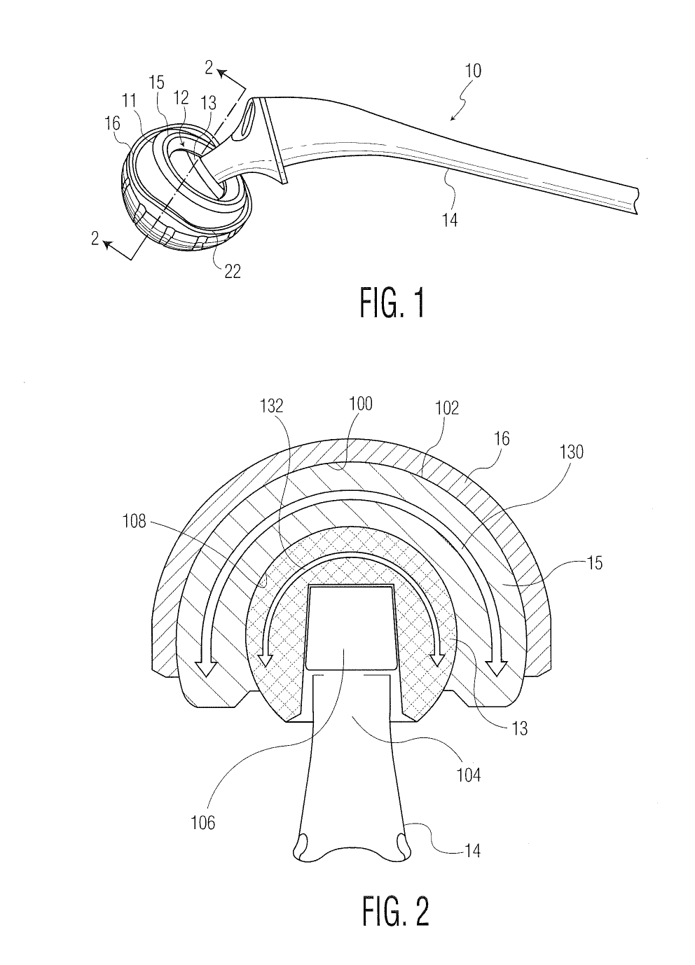

In the embodiment shown in FIGS. 1 and 2, the head 13 is mounted in and rotates in a UHMWPE first bearing component 15. First bearing component 15 in turn is free to rotate in PEEK shell 16. Shell 16 preferably has a polished inner surface 100 engaging the outer bearing surface 102 of polyethylene component 15. PEEK component 16 includes a rim 22 facing in a generally inferior direction which rim can either be planar or may have a curved portion profile to match the profile of the natural acetabulum. Referring to FIG. 2, it can be seen that the proximal end of femoral component 14 includes a neck section 104 and a trunion 106 which is typically conically tapered.

As can be seen in FIG. 2, there is shown a typical dual mobility acetabular cup system in which head 13 rotates on an inner bearing surface 108 UHMWPE inner bearing component 15. Component 15 outer bearing surface 102 in turn is freely rotatable on the inner bearing surface 100 of PEEK component 16. In the preferred embodiment, polyethylene bearing component 15 has been cross-linked in particular by the process described in U.S. Pat. Nos. 7,517,919, 7,714,036, 8,030,370 and 8,324,291 (hereinafter “X3 patents”).

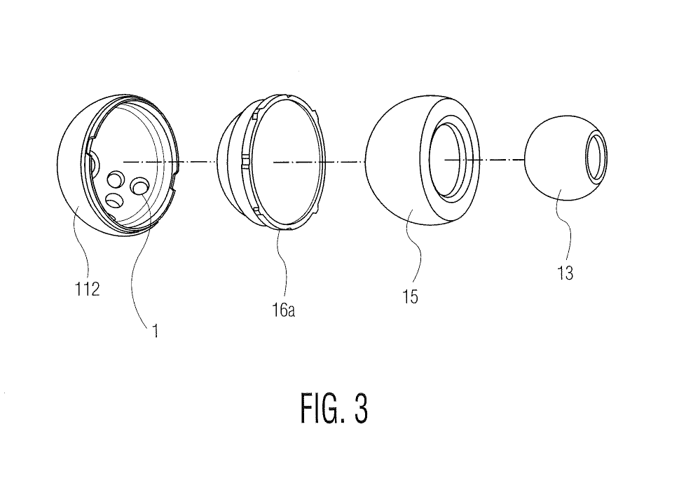

Referring to FIG. 3, there is shown an alternate embodiment of the dual mobility cup of the present invention which utilizes the same bearing system as shown in FIGS. 1 and 2 including a metal or ceramic head 13 and UHMWPE inner bearing 15 and the PEEK bearing component 16A which has an outer surface configured to attach to a metal outer shell 122. Outer shell 122 may be made of titanium or titanium alloy and have a tissue ingrowth surface 112 formed thereon.

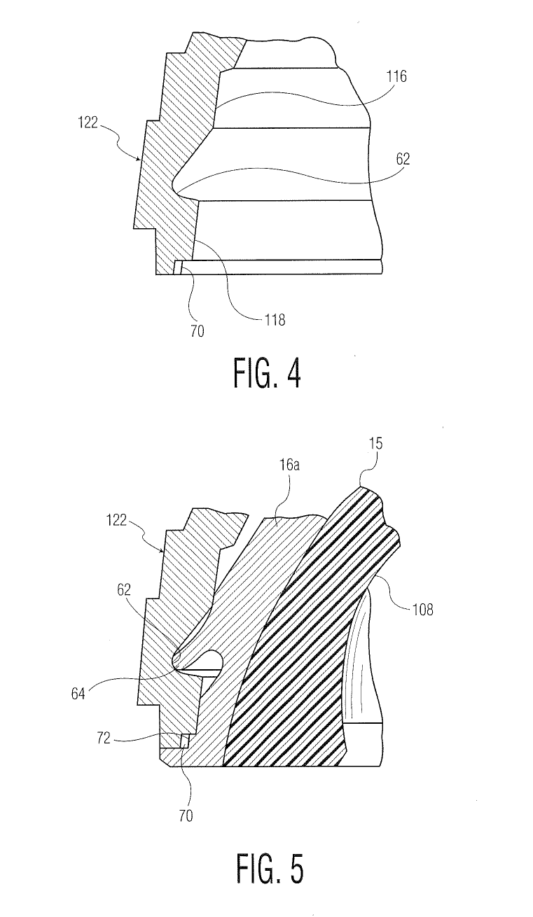

Referring to FIGS. 4 and 5, there is shown a typical metal outer shell generally denoted as 122 which may include tapered surfaces 116 and 118 separated by a lip receiving circumferential groove 62. An anti-rotation mechanism 70 is utilized to prevent rotation of the PEEK bearing member 16 about a polar axis of the acetabular cup system. Referring to FIG. 5 there is shown PEEK bearing component 16A held within metal outer shell 122 via a molded lip 64 which may snap into groove 62. An anti-rotation element 72 which mates with anti-rotation element 70 is formed on PEEK bearing component 16A to prevent the aforementioned rotation of bearing 16A within the acetabular cup shell 122. Such a system is taught in U.S. Pat. No. 6,475,243 which, however, does not teach the use of a dual mobility acetabular cup with a PEEK bearing component and a UHMWPE bearing component. However, the inner connection between the PEEK bearing component 16A and the outer metal shell 122 can be the same as that taught in U.S. Pat. No. 6,475,243 for the connection of a polyethylene bearing component to the metal outer shell. Likewise, the locking means 70, 72 which can be engagable protrusions and recesses formed on bearing components and 16A can be the same as described in U.S. Pat. No. 6,475,243.

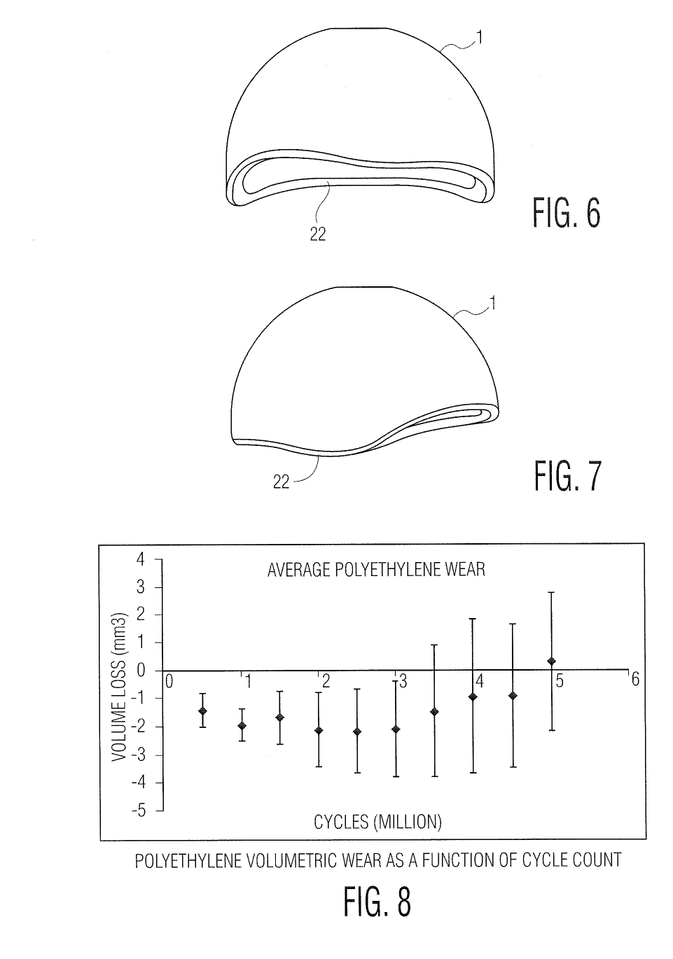

Referring to FIGS. 6 and 7, there is shown PEEK bearing component 16 including rim 22 which, as indicated contains a contour mimicking that of the natural acetabulum. Such a rim is described in U.S. Pat. No. 7,833,276, the disclosure which is incorporated herein by reference.

Referring to FIG. 8 there is shown the volumetric wear versus number of articulation cycles between the outer bearing surface 102 of UHMWPE bearing component 15 and the inner bearing surface 100 of PEEK bearing component 16 or 16A. The testing method for generating FIG. 8 will now be described by way of the following example.

### EXAMPLE

An initial wear characterization of PEEK acetabular liners against polyethylene dual mobility inserts was conducted to determine if there is any qualitative evidence of localized heating at that articular surface.

The PEEK acetabular liners tested had a 46 millimeter inner diameter. They were machined from extruded PEEK obtained from McMaster-Carr Company, N.J., USA and were polished to average surface roughness Ra Rod≦20. Four 28 millimeter inner diameter/46 millimeter outer diameter crosslinked UHMWPE dual mobility inserts were used for testing CoCr was the femoral head material. The crosslinking was as described in the X3 patents. The PEEK liners were cemented into metal acetabular shells for fixation purposes since the liner backsides did not seat completely flush against the shells part-spherical inner surface. A hip joint simulator (MTS, Eden Prairie, Minn.) was used for testing with the cups positioned anatomically (superior) oriented at 50° of inclination. Articulation was between surfaces 100 and 102 as indicated by arrow 130. Testing was run at 1 Hz with cyclic Paul curve physiologic loading applied axially, at a minimum of 2450 N. Samples were lubricated using Alpha Calf Fraction serum (Hyclone Labs, Logan, Utah) diluted to 50% with a pH-balanced 20-mMole solution of deionized water and EDTA (protein level≈20 g/l). Polyethylene wear was determined every 500,000 cycles gravimetrically. Simulated samples were soak corrected to account for any weight gain due to absorption of fluid. Weight loss data was converted to volumetric data and plotted as a function of cycle count. Testing was conducted for a total of 5 million cycles.

Average polyethylene volume loss plotted as a function of cycle count is shown in FIG. 8. The inner articular PEEK surface exhibited polishing and burnishing throughout the entire inner diameter and machining mark remnants on the outer diameter. The PEEK components exhibited scratches through the load path within its inner diameter, thus suggesting wear of the surface. At the completion of 5 million cycles there was no yellowing of the polyethylene. Yellowing of the polyethylene at the PEEK articular surface would suggest overheating of the polyethylene material. In the previous testing of PEEK on polyethylene samples in which polyethylene components overheated there was a distinct burning smell within the yellowed polyethylene area. This smell was not present with the samples tested.

The test results indicate only a low risk of polyethylene melting secondary to high friction and heat at the articular surface between dual mobility PEEK and polyethylene bearings under standard wear testing conditions. Polyethylene wear of this construct is very low and compares favorably to the wear results of dual mobility components utilizing CoCr as the second articular surface tested under the same conditions. Previous testing of 48 millimeter outer diameter X3 patent UHMWPE dual mobility components against COCr have a wear rate range of 1.9-0.59 mm³/mc, and 42 millimeter outer diameter dual mobility components have a wear rate range of 1.4-2.2 mm³/mc. This round testing of PEEK-on-X₃ UHMWPE dual mobility does show less UHMWPE wear than CoCr-on-X₃ UHMWPE dual mobility.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A total hip implant system comprising:
a femoral component comprising a part-spherical non-polymeric head having an outer bearing surface; and
a dual mobility acetabular cup system comprising an ultra-high molecular weight polyethylene first bearing component having a part-spherical inner bearing surface and a part-spherical outer bearing surface, the inner bearing surface articulating against the outer bearing surface of the part-spherical non-polymeric head, a second bearing component comprising neat non-fiber reinforced poly aryl ether ketone selected from the group consisting of polyetherketone (PEEK), polyetherether ketone (PEK), polyether ketone ketone (PEKK) and polyetherketone etherketoneketone (PEKEKK), the second bearing component having a part-spherical inner bearing surface articulating against the part-spherical outer bearing surface of the first bearing component.

2. The total hip system as set forth in claim 1 wherein the second bearing component has a bone contacting outer surface.

3. The total hip system as set forth in claim 2 wherein the second bearing component outer surface has a titanium mesh bone ingrowth surface embedded therein.

4. The total hip system as set forth in claim 1 wherein the dual mobility cup further comprises an outer metal shell with an outer bone contacting surface coupled to the second bearing component.

5. The total hip system as set forth in claim 4 wherein the second bearing component is fixedly mounted within the metal shell.

6. The total hip system as set forth in claim 5 wherein the outer metal shell has a circumferentially extending locking element formed on an inner surface for engaging a locking element formed on an outer surface of the second bearing component, the locking elements preventing relative movement between the outer shell and the second bearing component.

7. The total hip system as set forth in claim 1 wherein the part-spherical nonpolymeric head of the femoral component is made from a material selected from the group consisting of titanium, titanium alloy, ceramic, cobalt chrome molybdenum, and stainless steel.

8. The total hip system as set forth in claim 1 wherein the second bearing component has a circumferential distal rim having a generally inferiorly facing edge surface having a contour curved in a generally inferior-superior direction around the entire circumference of the distal rim with the generally inferiorly facing edge surfacing moving towards and away from a plane containing the rim of an acetabulum two times.

9. A total hip implant system comprising:
a prosthetic femoral component having a head with a part-spherical bearing surface; and
a dual mobility acetabular cup system comprising a first bearing component made of cross-linked ultra high molecular weight polyethylene mounted on the head, the first bearing component having a part-spherical inner bearing surface rotatably engaging the part-spherical bearing surface of the head, and having a part-spherical outer bearing surface, a second bearing component made of neat polyetheretherketone (PEEK) having an inner part-spherical bearing surface rotatably engaging the part-spherical outer bearing surface of the first bearing surface.

10. The total hip system as set forth in claim 9 wherein the second bearing component has a bone contacting outer surface.

11. The total hip system as set forth in claim 10 wherein the second bearing component outer surface has a titanium mesh bone ingrowth surface embedded therein.

12. The total hip system as set forth in claim 9 wherein the dual mobility cup further comprises an outer metal shell with an outer bone contacting surface coupled to the second bearing component.

13. The total hip system as set forth in claim 12 wherein the second bearing component is fixedly mounted within the metal shell.

14. The total hip system as set forth in claim 13 wherein the outer metal shell has a circumferentially extending locking element formed on an inner surface for engaging a locking element formed on an outer surface of the second bearing component, the locking elements preventing relative movement between the outer shell and the second bearing component.

15. The total hip system as set forth in claim 9 wherein the part-spherical nonpolymeric head of the femoral component is made from a material selected from the group consisting of titanium, titanium alloy, ceramic, cobalt chrome molybdenum, and stainless steel.

16. The total hip system as set forth in claim 9 wherein the second bearing component has a circumferential distal rim having a generally inferiorly facing edge surface having a contour curved in a generally inferior-superior direction around the entire circumference of the distal rim with the generally inferiorly facing edge surfacing moving towards and away from a plane containing the rim of an acetabulum two times.
